(19) United States
(12) Patent Application Publication
(10) Pub. No.: US 2006/0246417 A1
(43) Pub. Date: Nov. 2, 2006

(54) System for functional analysis of polypeptides

(76) Inventors: Ok-kyu Song, Pohang (KR); Sung Key Jang, Pohang (KR); Vit Kim, Pohang (KR); Joon Hyun Kim, Pohang (KR)

Correspondence Address: JHK LAW, P.O. BOX 1078, LA CANADA, CA, 91012-1078, US

(21) Appl. No.: 11/118,715

(22) Filed: Apr. 30, 2005

(51) Int. Cl. C12Q 1/00; C12N 1/18; C12N 5/06

(52) U.S. Cl. 435/4; 435/254.21; 435/325

(57) ABSTRACT

The present application discloses a polypeptide assay system that includes a non-mammalian cell in a non-mammalian cell culture medium expressing a heterologous polypeptide that is either displayed on its cell surface such that the polypeptide is the predominant polypeptide displayed on the cell surface or the polypeptide is secreted, and a target mammalian cell that includes a reporter construct in a mammalian cell culture medium.

### BACKGROUND OF THE INVENTION

1. Field of the Invention

The present invention relates to a systematic approach to expressing and analyzing protein ligands. The present invention also relates to a method for co-culturing non-mammalian cell expressing a heterologous polypeptide and target mammalian cell that contains a reporter responsive to the polypeptide so that the interaction between the heterologous polypeptide and the reporter or an element regulating expression of the reporter in the mammalian cell is assayed.

2. General Background and State of the Art

Cell surface display of heterologous protein was first accomplished by fusion of small proteins to the docking protein (pIII) of filamentous phage (Smith G P, 1985). Since then, other surface display systems have been developed and utilized in bacteria. However, yeast cells (i.e. Saccharomyces cerevisiae) are considered ideal for surface display systems, because 1) yeast is generally regarded as safe for use in food and pharmaceutical applications, 2) the yeast protein folding and secretory machineries are similar to those in mammalian cells, 3) well developed molecular engineering techniques are easily applicable to yeast cells, 4) yeast cells have rigid cell surfaces that should allow stable display of the target protein via a glycosyl phosphatidylinositol (GPI) anchor or disulfide bonds, and 5) unlike the case in E. coli, polypeptides produced in yeast can be post-translationally glycosylated during secretion through the ER and Golgi apparatus.

The GPI sequences of several glucanase-extractable proteins (e.g. the agglutinins Sag1 and Aga1, as well as Flo1, Sed1, Cwp1, Cwp2, Tip1, and Tir1) have been used to display heterologous proteins on the cell surfaces of yeast Saccharomyces cerevisiae. In addition, the signal sequences of secreted proteins have been combined with the GPI anchoring signal to direct the display of a normally secreted protein on the surface of yeast cells (Van der Vaart J M et al., 1997; Washida M. et al., 2001). Comparison of the incorporation capacity of the GPI anchoring sequences from several glucanase-extractable proteins revealed that the GPI anchoring sequence of Cwp2 can be used to effectively expose the immobilized protein on the surface of yeast cells (Van Der Vaart J M et al., 1997).

Various peptides and proteins, including the hepatitis B virus surface antigen, lipase, glucoamylase, α-galactosidase, green fluorescent protein (GFP) and single chain fragment (ScFv), have been displayed on the surfaces of yeast cells (Schreuder, M. P. et al. 1996, Boder, E. T. and Wittiup, K. D. 1997, Murai T. et al., 1997, Van Der Vaart J M et al., 1997, Ye et al., 2000, and Washida M. et al., 2001). These prior reports suggest that yeast surface display systems may be used as whole cell biocatalysts or live oral vaccines, as well as experimental platforms for the study of cell biology, regeneration of immobilized enzymes, immobilization of antibodies, and etc.

### SUMMARY OF THE INVENTION

The present invention is directed to a method for determining the function of a possible ligand activity of a polypeptide without purification of the polypeptide from the yeast cells producing the heterologous polypeptide, as follows:

For investigating the function of a protein, one of the two materials described below is added to a mammalian cell culture for functional testing of the ligand (i.e. testing for cytokine, chemokine, neurotransmitter, hormone, antibody or other activity).

A) Cell wall-bound protein: temperature sensitive non-mammalian cell, such as a yeast strain may be engineered to produce a protein of interest in a cell wall-bound form (FIGS. 1A and 1B). The heterologous gene may contain an N-terminal signal sequence and GPI anchoring sequence for attachment of the protein on the cell surface (FIG. 1A).

B) Secretory protein: temperature sensitive non-mammalian cell may be engineered to produce a protein of interest in secretory form (FIGS. 1C and 1D); mammalian cells may be co-cultured with the non-mammalian cell such as yeast or may be cultured in conditioned media from the non-mammalian cell culture. In the case of utilization of culture medium, a wild-type non-mammalian cell (rather than the temperature sensitive mutant) may be used for expression of the secretory protein. In this case, the protein of interest may be fused to a C-terminal signal sequence but not an anchoring sequence (FIG. 1C).

In the present application, the non-mammalian cell yeast is described and exemplified. However, it is to be understood that the invention is not limited to yeast. The yeast-expressed heterologous polypeptide utilized in this invention is generally referred to as a zymogand (zymogenic expressed ligand) and the system used in this invention is referred to as the zymogand system. The zymogand system may comprise several components, including:

A) An expression vector suitable for expression of a protein of interest in yeast, including either,

A-1) an expression vector for expression of cell wall-bound protein, containing a yeast promoter, a signal sequence for targeting the protein to the ER lumen, a sequence for integration of the secreted protein into the yeast cell wall, and an auxotrophic selection marker (FIG. 1A), or

A-2) an expression vector for expression of secretory proteins, containing a yeast promoter, a signal sequence for targeting the protein to the ER lumen, and an auxotrophic selection marker (FIG. 1C);

B) yeast cells capable of maintaining these expression vectors and producing the encoded heterologous proteins, including either,

B-1) temperature sensitive (or other conditionally growing) yeast cells producing cell wall-bound proteins (FIG. 1B), or

B-2) wild-type yeast cells producing secretory proteins (FIG. 1D); and

C) mammalian cells suitable for measuring the bio-activity of the yeast-expressed polypeptides.

With this method, systematic analysis of protein ligand activities of putative genes is possible at the genomic level. A secretory or surface-displayable fusion protein is expressed in continuously or conditionally growing yeast cells (or other unicellular organisms) through the use of fusion gene, and tested for its ability to function as an actual ligand to affect a mammalian cell via co-cultivation of yeast and mammalian cells, or cultivation of mammalian cells in conditioned media from the yeast cells.

Thus, the present invention is directed to a polypeptide assay system comprising: (1) a non-mammalian cell in a non-mammalian cell culture medium expressing a heterologous polypeptide that is either displayed on its cell surface such that the polypeptide is the predominant polypeptide displayed on the cell surface or the polypeptide is secreted; and (2) a target mammalian cell comprising a reporter construct in a mammalian cell culture medium. In this assay system, the non-mammalian cell culture medium may not be suitable for culturing mammalian cell, and the mammalian cell culture medium may be suitable for culturing mammalian and non-mammalian cell. Further, the non-mammalian cell and the mammalian cell may be mixed together. Still further, the non-mammalian cell may be a fungal cell or prokaryotic cell, and the fungal cell may be yeast cell such as those belonging to the genus Saccharomyces. In the assay system, the non-mammalian cell may be also a conditional mutant, such as a temperature sensitive mutant. The mammalian cell is preferably a human cell.

In another aspect, the present invention is also directed to a method of assaying for the function of a polypeptide comprising: (a) culturing a non-mammalian cell expressing a heterologous polypeptide in a non-mammalian cell culture medium so that the polypeptide is displayed on the cell surface such that the polypeptide is the predominant polypeptide displayed on the cell surface; (b) culturing a target mammalian cell comprising a reporter construct in a mammalian cell culture medium; (c) mixing the non-mammalian cell culture in (a) with the mammalian cell culture in (b), wherein a change in expression of the reporter construct in the mammalian cell indicates that the heterologous polypeptide is a modulator of the reporter. In this method, the non-mammalian cell culture medium may not be suitable for culturing mammalian cell, and the mammalian cell culture medium may be suitable for culturing mammalian and non-mammalian cell. Further, the non-mammalian cell may be a fungal cell or prokaryotic cell. The non-mammalian cell may be a yeast cell such as those belonging to the genus Saccharomyces. The non-mammalian cell may be a conditional mutant such as a temperature sensitive mutant. Further in the method described above, the temperature of the mixed culture medium may be modified so that the mammalian cell grows but the non-mammalian cell does not grow in the medium.

In yet another embodiment of the invention, the invention is directed to a method of assaying for the function of a polypeptide comprising: (a) culturing a non-mammalian cell expressing a heterologous polypeptide in a culture medium so that the polypeptide is secreted; (b) culturing a target mammalian cell comprising a reporter construct; (c) mixing the non-mammalian cell culture medium comprising the secreted polypeptide in (a) with the mammalian cell culture in (b), wherein a change in expression of the reporter construct in the mammalian cell indicates that the heterologous polypeptide is a modulator of the reporter. In this method, the non-mammalian cell may be a fungal cell or prokaryotic cell. The fungal cell may be a yeast cell such as those belonging to the genus Saccharomyces.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1D show a schematic diagram of the zymogand analysis system. A mammalian gene is heterologously expressed in yeast cells either as a cell wall-bound (FIGS. 1A and 1B) or secretory (FIGS. 1C and 1D) form. (A) Schematic diagram of a fusion gene encoding a cell wall-bound zymogand. The mammalian protein is expressed in yeast by introduction of a high copy yeast shuttle expression vector encoding a fusion protein in which the mammalian sequence is flanked with the N-terminal part of the Cwp2 protein (signal sequence) and the C-terminal part of the Cwp2 protein, which directly anchors the mammalian protein to the yeast cell wall (Ram et al., 1998). To facilitate fusion protein expression, the translation termination codon of the mammalian gene is deleted and the codon encoding the last amino acid of the target protein is fused in-frame with the C-terminal part of Cwp2. (B) Temperature sensitive yeast cells (PBN404) with surface expression of zymogands were then incubated with mammalian cells at 37° C. for examination of their effects on mammalian cells. The effect can be monitored by using a variety of techniques depending on the reporter system that is used. (C) A schematic diagram of a fusion gene encoding a secretory zymogand. The utilized yeast expression vector encodes the N-terminal part of the Cwp2 protein (signal sequence) followed by the mammalian sequence, in which the translation termination codon is maintained. (D) Zymogand-secreting yeast cells are incubated with mammalian cells at 37° C., or alternatively, yeast cells are incubated in mammalian cell culture medium, which is then filtered and added to cultured mammalian cells.

FIG. 2 shows amounts of secretory TNF-α (zymo-sTNF-α) secreted from yeast cells. The amounts of zymo-sTNF-α secreted in the medium were measured by Western blot analysis using an antibody against TNF-α (Roche). Yeast cells (3×10⁷) at mid-log phase were washed with phosphate buffered saline (PBS) and resuspended in 1 ml of DMEM. The yeast cell suspension was incubated at 37° C. for 2 h, and the medium was collected by filtration with a membrane filter (0.2 μm pore). Western blotting was performed on 20 μl aliquots of media cultivated with yeast cells containing control vector p423GPD (lane 1), plasmid p423-bTNF-α expressing cell wall-bound zymo-bTNF-α (lane 2), and plasmid p423-sTNF-α expressing secretory zymo-sTNF-α (lane 3). As a reference, purified TNF-α protein (Roche) was applied at concentrations of 0.2, 0.3, 0.5, and 1.0 ng in lanes 5, 6, 7, and 8, respectively. About 0.8 ng of zymo-sTNF-α was secreted from 6×10⁵ yeast cells containing plasmid p423-sTNF-α during a 2 h incubation (lane 3). Soluble TNF-α protein was not detected in the medium cultivated with yeast cells containing control vector (lane 1) or p423-bTNF-α (lane 2).

FIG. 3 shows the effect of zymo-sTNF-α-secreting yeasts on expression of a reporter gene (firefly luciferase) under the control of a NF-κB-responsive element. 293T cells (3×10⁵) harboring plasmids PNF-κB (Stratagene), which contains a firefly luciferase gene under the control of a NF-κB-responsive element, and pRL-CMV (Promega), which contains a Renilla luciferase gene under control of the CMV promoter, were treated for 12 hours with 10 ng of TNF-α (lane 2), or 3×10⁵ (lanes 3 and 5) and 6×10⁵ (lanes 4 and 6) yeast cells containing control vector (lanes 3 and 4) or the expression vector for the secretory form of TNF-α. Cells were harvested and lysed, and the luciferase activity of the lysates was measured. The bars indicate relative luciferase activity, with the activity of control (untreated) cells (indicated as Mock in the figure) set to 1 (lane 1).

FIG. 4 shows the effect of conditioned yeast media containing secreted zymo-sTNF-α on cultured mammalian cells. Yeast cells containing various plasmids (pGAL4, p423GAL1 and p423-sTNF) were cultivated to mid-log phase in synthetic complete media (lacking leucine) and harvested. The yeast cells (3×10⁷) were washed with phosphate buffered saline (PBS), resuspended in 1 ml DMEM, and incubated at 37° C. for 2 h. The medium was collected by filtration with a membrane filter (0.2 μm pore). Five μl (lanes 2 and 7), 10 μl (lanes 3 and 8), 20 μl (lanes 4 and 9), 40 μl (lanes 5 and 10), and 80 μl (lanes 6 and 11) conditioned media, or 0.2 ng (lane 12), 0.4 ng (lane 13), 0.8 ng (lane 14), 1.6 ng (lane 15) and 3.2 ng (lane 16) purified TNF-α were added to culture medium of 293T cells (3×10⁵ cells) containing plasmids pNFκB and pRL-CMV. The 293T cells were cultivated for 12 h at 37° C. Cells were harvested and lysed, and the lysate luciferase activities were measured. The bars indicate relative luciferase activity in the cells after treatment with TNF-α or conditioned media, with the luciferase activity in control (untreated) cells (indicated as Mock in the figure) set to 1 (lane 1).

FIGS. 5A-5B show morphology of yeast and mammalian cells. (A) Comparison of yeast and mammalian cells. HeLa/E cells treated with yeast cells grown to mid-log stage in YEPD were fixed with 3.5% (W/V) paraformaldehyde (Sigma) at room temperature for 12 min and washed three times with PBS. The samples were stained with 0.5% Fluorescent Brightener 28 (Sigma) for 30 min at room temperature. The yeast cells were confirmed by Differential Interference Contrast (DIC) imaging and yeast-specific staining with fluorescent brightener 28 (Sigma). The yeast cells were visualized in blue at bottom left of the picture. (B) Expressions of interferon-α on the surface of yeast and interferon-α/β receptor on the surface of HeLa cell. HeLa/E cells were grown on coverslips coated with 0.2% gelatin for 48 h and then washed three times with PBS. The cells were fixed with 3.5% (W/V) paraformaldehyde (Sigma) at room temperature for 12 min, and washed three times with PBS. The samples were soaked in blocking solution (PBS containing 1% BSA) for 30 min at room temperature (RT), incubated with anti-IFN-α/β receptor antibody (Santa Cruz Biotechnology) for 1 hr at RT, and then washed three times with PBS. Samples were treated with fluorescein isothiocyanate (FITC)-conjugated secondary antibodies (Jackson ImmunoResearch Laboratories) at RT for 1 hr. Yeast cells were grown to mid-log stage in YEPD and were fixed with 3.5% (W/V) paraformaldehyde (Sigma) at RT for 12 min and washed three times with PBS. The samples were stained with 0.5% Fluorescent Brightener 28 (Sigma) for 30 min at RT, incubated with the primary antibody (anti-IFN-α antibody; Santa Cruz Biotechnology) for 1 h at RT, and then washed with PBS three times. Samples were then treated with TRITC-conjugated secondary antibody (Jackson ImmunoResearch Laboratories) for 1 h at RT. Finally, the coverslips were washed three times with PBS, placed on glass slides and sealed with transparent nail polish. The fluorescent images were captured with a cooled CCD camera and Zeiss Axioplan microscope. Data were processed using the Adobe Photoshop software. The IFN-α receptors and IFN-α are visualized as green and red dots respectively. The yeast and HeLa cell images were generated separately and then combined together for comparison.

FIG. 6 shows the antiviral effects of zymo-interferon-α (zymo-IFN-α) against hepatitis C virus (HCV). Plasmids p425-sINF-α (expressing secretory zymo-sIFN-α) and p425-bINF-α (expressing cell wall-bound zymo-bIFN-α) were transformed into yeast PBN404. Huh-7 human hepatocyte cells containing a subgenomic HCV replicon RNA with a reporter Renilla luciferase (assayable replicon RNA; Bartenschlager, 2002), which can be used to assay changes in HCV RNA levels (Vrolijk et al., 2003), were used to monitor the anti-HCV effects of zymo-bIFN-α. (A) The effect of purified IFN-α protein on the HCV replicon. Huh-7 cells (1.5×10⁴ cells) containing the assayable HCV subgenomic replicon RNA were treated with 0, 10, 20, 40, 80, and 160 international units (IU) of purified IFN-α (Calbiochem); the respective Renella luciferase activities are shown in lanes Mock, 10 IU, 20 IU, 40 IU, 80 IU, and 160 IU, respectively, with that of the Mock-treated lysate set at 100%. (B) The effect of zymo-sIFN-α-secreting yeast cells on HCV replication. Huh-7 cells (1.5×10⁴ cells) containing the assayable HCV subgenomic replicon RNA were treated with 0, 2.5×10³, 5×10³, 1.0×10⁴, 2.0×10⁴, and 4.0×10⁴ yeast cells containing plasmid p425-sINF-α, cultured for 24 h, and then assayed for Renilla luciferase activity as shown in lanes Mock, 2.5, 5, 10, 20, and 40, respectively. The bars indicate relative luciferase activities, with that of the Mock-treated lysate set at 100%. (C) Effect of cell wall-bound zymo-bIFN-α-producing yeast cells on HCV replication. Experiments were carried out as in (B), utilizing yeast cells containing plasmid p425-bINF-α. Cell wall-bound zymo-bINF-α showed a higher antiviral activity than did secretory zymo-sIFN-α; compare panel (C) with (B). (D) Effect of control yeast cells on HCV replication. Experiments were carried out as in (B), utilizing yeast cells containing negative control plasmid p425. No antiviral activity was observed in lysates from the control yeast cells.

FIGS. 7A-7E show anti-hepatitis C virus (HCV) effects of yeast cells producing zymo-ligands, including zymo-interferon-γ (zymo-IFN-γ), zymo-TNF-α and zymo-transforming growth factor-β (zymo-TGF-β). Yeast cells producing zymo-sIFN-γ, zymo-bIFN-γ and TGF-β were generated by transforming yeast PBN404 with plasmids p425-sINF-γ (secreted), p425-bINF-γ (cell wall-bound) and p425-sTGF-β (secreted), respectively. Huh-7 cells containing a subgenomic HCV replicon RNA with a reporter Renilla luciferase (described above) was used to monitor the antiviral effects of the zymogands. (A) Effect of negative control yeasts containing parental plasmid p425GPD. Huh-7 cells (1.5×10⁴ cells) containing the assayable HCV subgenomic replicon RNA were co-cultured with 0, 2.5×10³, 5×10³, 1.0×10⁴, 2.0×10⁴, and 4.0×10⁴ yeast cells expressing the control p425 plasmid, and samples were assayed for Renilla luciferase activity as shown in lanes Mock, 2.5, 5, 10, 20, and 40, respectively. The bars indicate the relative luciferase activities, with that of the Mock-treated lysate set at 100%. (B) Effect of yeast cells producing cell wall-bound zymo-IFN-γ on HCV replication. Experiments were carried out as in (A), utilizing cells harboring plasmid p425-bINF-γ. Cell wall-bound zymo-INF-γ showed a weak anti-HCV activity. (C) Effect of yeast cells producing cell secretory zymo-sIFN-γ on HCV replication. Experiments were carried out as in (A), utilizing plasmid p425-sINF-γ. No anti-HCV effect was observed under the tested conditions. (D) Effect of yeast cells producing secretory zymo-TNF-α on HCV replication. Experiments were carried out as in (A), utilizing plasmid p425-sTNF-α. No anti-HCV effect was observed under the tested conditions. (E) Effect of yeast cells producing secretory zymo-TGF-β on HCV replication. Experiments were conducted as in (A), utilizing plasmid p425-sTGF-β. No anti-HCV effect was observed under the tested conditions.

FIG. 8 shows Zymo-sTGF-β induces phosphorylation of Erk protein. Yeast cells (3×10⁷) containing plasmid p425 (lane 1), p425-bTGF-β (lane 2), or p425-sTGF-β (lane 3) at mid-log phase were harvested and incubated at 37° C. for 2 h, and the heat-treated yeast cells (1.0×10⁵) were applied to the culture media of 3×10⁴ RINm5F cells (Rat Insulinoma) for the indicated times. Purified epidermal growth factor (EGF) was used as the positive control (lane 4). Treated cells were harvested and lysed, and the levels of phosphorylated Erk protein were examined by Western blotting using an antibody against a phospho-Erk oligopeptide. Phosphorylated Erk protein was detected in RINm5F cells treated with yeast cells secreting zymo-TGF-β for 2 and 5 min.

### DETAILED DESCRIPTION OF THE PREFERRED EMBODIMENTS

In the present application, “a” and “an” are used to refer to both single and a plurality of objects.

As used herein, “cell surface display” and “cell-surface expression” refer to a protein or peptide that is linked to an appropriate anchoring motif. The display may be based on expression of a heterologous polypeptide fused to anchoring motifs that direct their incorporation on the cell surface. The recombinant protein fused to the anchoring motif, which is expressed in the cytosol of the host cell, may be transported across the cell wall and membrane with the guide of the anchoring motif. Cell surface display allows the peptides and proteins to be displayed on the outer surface of the cells. The polypeptide to be displayed can be fused to an anchoring motif by N-terminal fusion, C-terminal fusion or sandwich fusion.

As used herein, “chimeric” refers to the combination of two domains.

As used herein, “conditional mutant” refers to a mutant mammalian or non-mammalian cell that does not grow under a particular environmental conditions in which normal cells are usually unaffected. An example is temperature-sensitive mutant yeast cell that does not grow at a certain temperature that is suitable for growth for normal yeast cells. Other conditional mutants may include those that are sensitive to other environmental factors such as pH, salt conditions and so forth.

As used herein, “displayed” refers to exposure of polypeptides that are transported across the cell membrane to the extracellular environment by anchoring to the surface of the cell expressing the gene encoding the polypeptide.

As used herein, “fusion protein” refers to a protein created by expression of a hybrid gene made by combining two gene sequences. Typically this is accomplished by cloning a cDNA into an expression vector in frame with an existing gene. Such fusion gene may include an anchoring protein and a heterologous polypeptide such that the heterologous polypeptide is displayed on the outer surface of the cell.

As used herein, “GPI anchoring sequence” refers to the sequences found in glycosylphosphatidylinisotol (GPI) anchored proteins such as agglutinins Sag1 and Aga1, Flo1, Sed1, Cwp1, Cwp2, Tip1, and Tir1. The signal for GPI-anchoring is typically confined to the C-terminus of the target protein. GPI anchored proteins are preferably linked at their carboxyterminus through a phosphodiester linkage of phosphoethanolamine to a trimannosyl-non-acetylated glucosamine (Man3-GlcN) core. The reducing end of GlcN is linked to phosphatidylinositol (PI). PI may then be anchored through another phosphodiester linkage to the cell membrane through its hydrophobic region. Intermediate forms may be also present in high concentrations in microsomal preparations. Fusion of the GPI anchoring sequence with a gene allows the fused gene product or the encoded protein to be displayed on the surface of the cell expressing the fusion construct.

As used herein, “heterologous protein” refers to non-native protein produced by a host cell.

As used herein, “ligand” or “protein ligand” refers to any molecule or polypeptide molecule that binds to its specific binding partner including a receptor protein. A ligand may bind to its receptor protein to form a complex. The ligand may be an agonist or an antagonist, and may stimulate or inhibit an activity by its binding.

As used herein, “mammalian” refers to the common name for the warm-blooded animals, which include humans and any other animal that nourishes its young with milk, has hair, and has a muscular diaphragm. Mammalian also includes, but is not limited to, rats, mouse, pigs, and primates, including humans.

As used herein, “medium” or “media” refers to the growth medium or culture medium, which is usually in solution form and free of all contaminant microorganisms by sterilization and containing the substances required for the growth of cells or organisms such as bacteria, protozoans, algae, fungi, plants, and mammalian cells. Some media consist of complex ingredients such as extracts of plant or animal tissue (e.g., peptone, meat extract, yeast extract); others contain exact quantities of known inorganic salts and one or more organic compounds (synthetic or chemically defined media). Various types of living cells, or tissue cultures, also may be used as media. Dividing cells from various mammalian tissues can be grown in vitro under careful laboratory control. “Mammalian cell culture medium” refers to medium that is prepared to be suitable specifically for growth of mammalian cells by including all of the ingredients that are required for mammalian cell growth.

As used herein, “modulator” refers to a polypeptide that affects gene expression or protein regulation in the target mammalian cell. The modulator may bind its target cell via a receptor molecule on the target cell surface. This interaction may trigger a cascade of signals within the target cell that alters the target cell's gene expression or protein regulation. The modulator may up-regulate and stimulate physiologic activity or it may down-regulate and inhibit physiologic activity through gene regulation or at the protein level.

As used herein, “non-mammalian” refers to all living organisms excluding mammalian organisms. Non-mammalian organisms include, but not limited to, fungi and bacteria. Fungi include without limitation yeast such as those belonging to the genus Saccharomyces including, but not limited to, Saccharomyces cerevisiae and Schizosaccharomyces pombe and other types of yeast such as Candida albicans. Bacteria include genera Pseudomonas, Staphylococcus, Bacillus, and Escherichia, including E. coli.

As used herein, “polypeptide” refers to any polypeptide that is displayed or secreted by the non-mammalian cells. Any polypeptide that is desired to be tested for its effects on a target cell may be used. Thus, the present invention is not limited by any particular polypeptide or type of polypeptide so long as the polypeptide is capable of being expressed in a non-mammalian cell and is able to be displayed on the cell surface or secreted. The various polypeptides may include, but not limited to, virus surface antigen, lipase, glucoamylase, α-galactosidase, green fluorescent protein (GFP), single chain fragment (ScFv), cytokine, neurotransmitter, hormone, and antibody.

As used herein, “predominant” refers to a large amount of a heterologous polypeptide, which is expressed and displayed on the cell surface as compared to the endogenous proteins or polypeptides that may be present on the cell surface. By predominant, at least 30% of the displayed polypeptides is contemplated. Further, at least 40%, 50%, 60%, 70%, 80%, or 90% of the displayed polypeptides on the cell surface may be considered to be predominant.

As used herein, “reporter” refers to a gene or protein. In the case of a gene construct, a transcriptional regulatory element is linked to the gene encoding the reporter protein. The reporter can be a coding sequence attached to heterologous promoter or other gene regulatory element and whose product is easily and quantifiably assayed when the reporter construct is introduced into tissues or cells. The “reporter” also refers to a receptor that a ligand expressed heterologously from the non-mammalian cell may bind so that the complex of the ligand/reporter may be visualized such as by antibody precipitation.

As used herein, “target cell” refers to the mammalian cell containing reporting elements.

As used herein, “temperature sensitive mutant” refers to an organism that has a wild-type phenotype at a permissive temperature but a mutant phenotype at a restrictive or non-permissive temperature. In an exemplified version of a temperature sensitive yeast cell, the yeast may grow normally at 30° C. However, it may cease to grow at 37° C. Other types of environmentally sensitive non-mammalian mutant strains such as pH sensitive or resistant organism may also be used in the practice of the invention.

As used herein, “yeast expressed mammalian ligand” refers to protein molecule produced from yeast cell with a vector expressing a gene of mammalian origin.

As used herein, “zymogand” refers to the yeast-expressed mammalian ligand.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

1. Testing of Membrane-Bound Zymogands

Yeast (PBN404) cells were cultivated in synthetic complete media (lacking specific amino acids as necessary for plasmid maintenance) overnight at 30° C. The resulting cultures were diluted to an optical density (OD)₆₀₀ of 0.4, further grown to an OD₆₀₀=1.3, and then harvested by centrifugation at 1500×g for 5 min. Yeast cells were re-suspended in mammalian cell culture medium (1 ml of DMEM), cultivated at 37° C. for 2 h, and then cultured with mammalian cells capable of responding to the yeast-expressed ligand. The yeast and mammalian cells were co-cultivated at 37° C. for 1 min to several days, depending on the utilized reporter, and zymogand activity was monitored by various assay systems, including but not limited to:

monitoring the up- or downregulation of a gene controlled by a ligand-regulated promoter,

measuring the viral genome copy levels (DNA or RNA) or expression of a reporter gene under the control of a virus gene expression system (for testing of antiviral effectiveness),

examination of the phosphorylation or dephosphorylation of a protein known to specifically mediate a ligand-specific signaling cascade, and

assaying cytosolic release of secondary messengers, i.e. calcium, which can be measured by intensity changes of a calcium-interacting fluorescein.

2. Testing of Secretory Zymogands

Yeast cells were cultivated in synthetic complete media lacking the appropriate amino acids overnight at 30° C. The resulting cultures were diluted to an OD₆₀₀=0.4, further grown to an OD₆₀₀=1.3, and harvested by centrifugation at 1500×g for 5 min. The yeast cells were re-suspended in mammalian cell culture medium (1 ml of DMEM), cultivated at 37° C. for 2 h, and the yeast culture medium was recovered by filtration through a Millipore filter (0.2 μm). The conditioned medium was then added to mammalian cell culture medium containing mammalian cells harboring the appropriate reporter gene. Alternatively, yeast cells expressing the secretory proteins were adapted at 37° C. for 2 h and then added directly to the mammalian cell culture. The mammalian cells were cultivated further at 37° C. for 1 min to several days, depending on the utilized reporter, and zymogand activity was monitored as above.

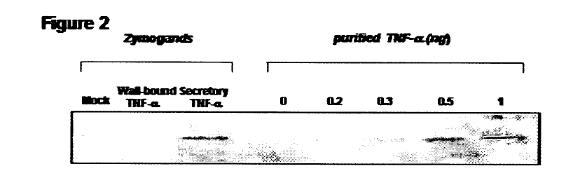

In order to produce zymogands, we utilized Cwp2, a major cell wall mannoprotein, as a carrier protein. Here, we tested our strategy of yeast surface presentation and/or secretion of ligands and of using the whole yeast cell as a functional ligand supply by using human IFN-α, human IFN-γ, human TGF-β3, and human TNF-α as model zymogands. This method has the advantage of using direct co-cultivation of yeast and mammalian cells, and requiring no additional purification of the yeast-expressed fusion protein. In order to minimize the effects of the yeast cells on the mammalian cell cultures, we generated a temperature sensitive yeast strain, named PBN404 [MATa, ura-52, his3-200, ade2-101::pGAL2-ADE2 trp1-901, leu2-3,112, gal4d, gal80d, met-,ura3::kanMX6-pGAL1-URA3::pGAL1-lacZ], which grows at 30° C. but not 37° C., allowing co-incubation of the yeast and mammalian cells at 37° C. for more than 24 h without deleterious effects such as nutrient depletion or secretion of toxic materials by growing yeast cells. Interestingly, the production and secretion of zymogands by the existing yeast cells continued at 37° C. in the mammalian culture media (FIG. 2). For instance, about 0.8 ng of zymo-TNF-α was secreted into the culture media from 6×10⁵ yeast cells during a 2 h incubation, as estimated by Western blotting (FIG. 2).

### Example 2

### Effect of zymo-TNF-α-Producing Yeast on Expression of a Reporter Gene Under the Control of a NF-κB Responsive Element

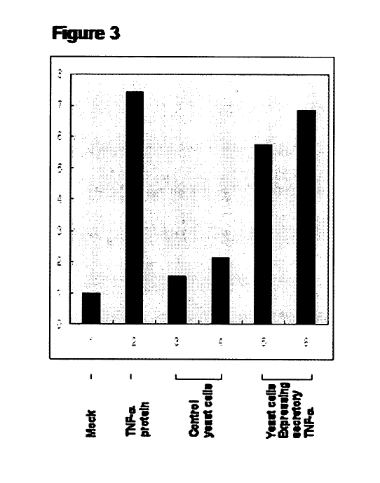

In order to test whether zymogand activity can be measured by co-cultivation of mammalian and yeast cells, we co-cultivated 293T cells transfected with plasmids PNFκB and pRL-CMV with zymo-sTNF-α-producing yeast cells at 37° C. for 12 h. Co-culture of the 293T cells with yeast producing zymo-sTNF-α induced strong reporter (luciferase) activity in mammalian cells (FIG. 3, lanes 5 and 6), comparable to that induced by 10 ng of purified TNF-α (FIG. 3, lane 2), while co-culture with yeast cells harboring the control plasmid induced only marginal reporter activation (FIG. 3, lanes 3 and 4). This indicates that the effects of mammalian proteins can be monitored by co-cultivation of the expressing yeast with mammalian cells containing a proper reporter gene.

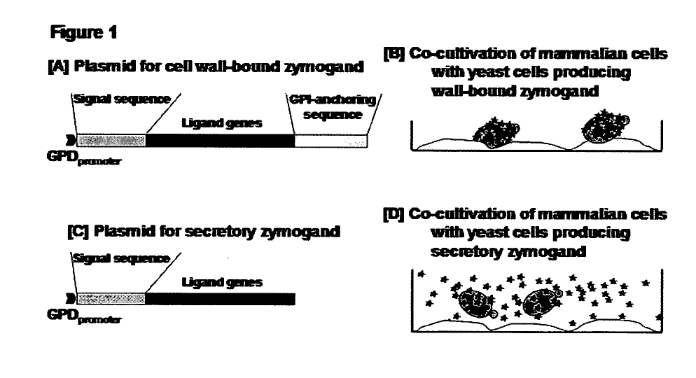

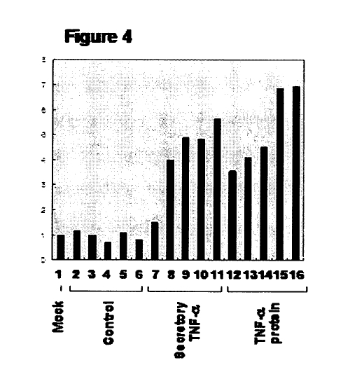

We then tested the effect of secreted zymo-sTNF-α on the NFκB response element by culturing mammalian cells with conditioned medium from yeast producing secretory zymo-sTNF-α (FIG. 4). Yeast cells (3×10⁷) at mid-log phase were washed with phosphate buffered saline (PBS), resuspended in 1 ml of DMEM, and incubated at 37° C. for 2 h. The medium was collected by filtration with a membrane filter (pore size, 0.2 μm), and the filtrates were added to cultured 293T cells harboring plasmids pNF-κB and pRL-CMV. Cells were incubated at 37° C. for 12 h, and cell lysates were prepared and assayed for firefly and Renilla luciferase activities, which reflected NF-κB activation and DNA transfection efficiency, respectively. The levels of NF-κB activation normalized against transfection efficiency are shown in FIG. 4. The conditioned media from yeasts expressing secretory zymo-sTNF-α strongly activated the reporter in a dose-dependent manner (FIG. 4), indicating that this strategy can be utilized for examining the function of a secreted zymogand (FIG. 1C). This method has the benefit of not requiring temperature sensitive yeast cells, since there is no co-cultivation step (data not shown).

### Example 3

### Antiviral Effects of Yeast Cells Producing Zymogands

Many cell membrane-bound protein ligands trigger signaling cascades through interactions with receptors on the surface of target cells. We tried to mimic this situation by producing zymogands in a cell wall-bound form. As model systems, we examined the antiviral effect of secretory (zymo-sIFN-α) and cell wall-bound (zymo-bIFN-α) IFN-α in a Huh-7 human hepatocarcinoma cell line containing a hepatitis C viral replicon. This system mimics replication cycle of hepatitis C virus (HCV) (Bartenschlager, 2002) and can be assayed via a Renilla luciferase reporter gene (assayable replicon RNA; Bartenschlager, 2002).

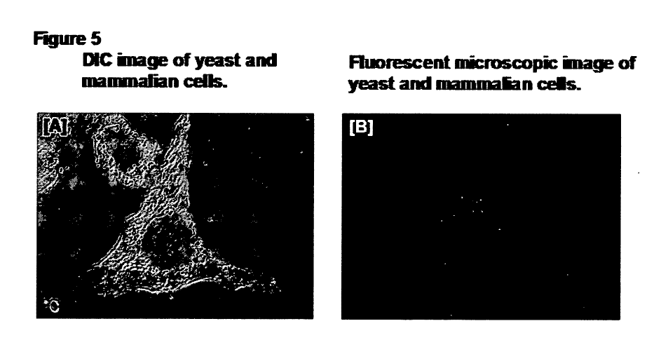

The expression of IFN-α on the surface of yeast cells and IFN-α/β receptors on the surface of Huh-7 cells was monitored by immunocytochemistry. The yeast cells were confirmed by Differential Interference Contrast (DIC) imaging and yeast-specific staining with fluorescent brightener 28 (Sigma) (blue cell in FIG. 5A). For visualization of yeast surface-bound IFN-α, unpermeablized yeast cells were treated with an anti-IFN-α antibody (Santa Cruz Biotechnology) and a TRITC-conjugated secondary antibody (Jackson ImmunoResearch Laboratories) (red signal, bottom left corner of FIG. 5B). The whole surface of yeast glowed in red, indicating that heterologous genes can be expressed and presented on the surface of yeast cells using the system described in FIGS. 1A and 1B. For visualization of surface-expressed IFN-α/β receptors, unpermeablized HeLa cells were treated with a primary antibody against IFN-α/β receptors (Santa Cruz Biotechnology) and a FITC-conjugated secondary antibody (Jackson ImmunoResearch Laboratories). IFN-α/β receptors were observed in punctate clusters on the surface of Huh-7 cells (green dots in FIG. 5B).

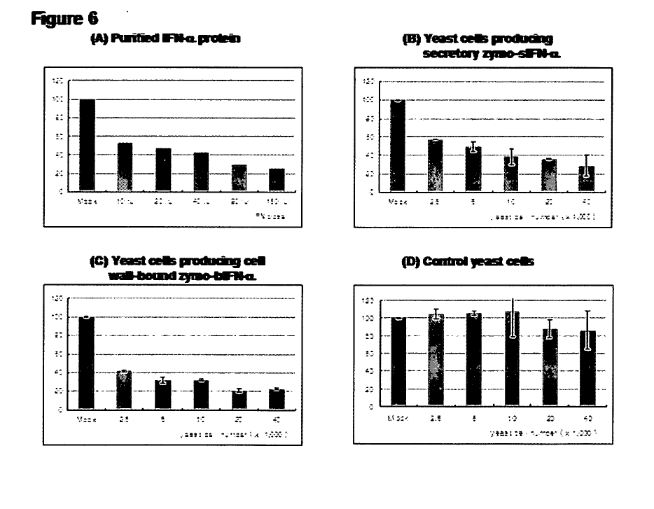

Purified INF-α (positive control) inhibited proliferation of HCV replicon RNA in Huh-7 cells in a dose-dependent manner (FIG. 6A), indicating that the utilized cell-based assay system was suitable for measuring the anti-HCV effects of IFN-α. Similarly, co-culture with yeasts producing zymo-sIFN-α (FIG. 6B) and zymo-bIFN-α (FIG. 6C) both inhibited the proliferation of replicon RNAs in Huh-7 cells in a dose-dependent manner, while yeast cells expressing the control plasmid did not (FIG. 6D). Interestingly, yeasts producing cell wall-bound zymo-bIFN-α showed a higher antiviral activity than those producing secretory zymo-sIFN-α. The molecular basis of this difference remains to be elucidated.

### Example 4

### Anti-HCV Effects of Various Zymogands

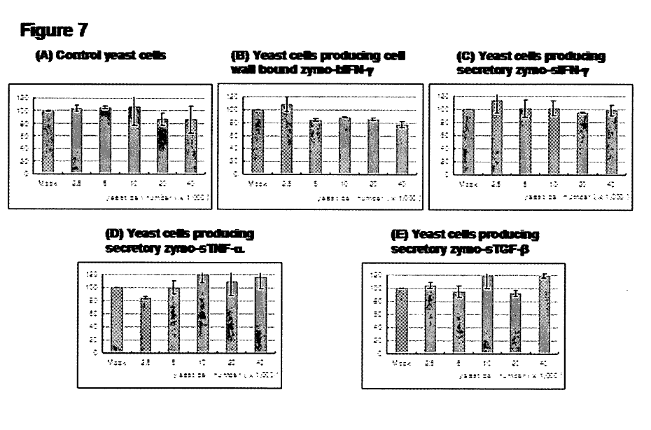

in order to test the effect of various zymogands on proliferation of the HCV replicon, yeasts cells producing cell wall-bound interferon-γ (zymo-bIFN-γ), secretory interferon-γ(zymo-sIFN-γ), secretory tumor necrosis factor-α (zymo-sTNF-α), and secretory transforming growth factor-β (zymo-sTGF-β) were generated using the plasmids described in FIG. 1. Yeasts producing zymo-bIFN-γ showed a weak antiviral effect (FIG. 7B) that was much lower than that of IFN-α (FIG. 6B and 6C), but consistent with that of purified IFN-γ (data not shown). No antiviral activity was observed from control yeasts (FIG. 7A) and those producing zymo-sIFN-γ, zymo-TNF-α, and zymo-sTGF-β (FIGS. 7C, 7D, and 7E, respectively). These results indicate that the antiviral effects of proteins can be tested using the inventive yeast-based system.

### Example 5

### Measuring Mitogenic Signal Cascade Activation by Observing Erk Protein Phosphorylation

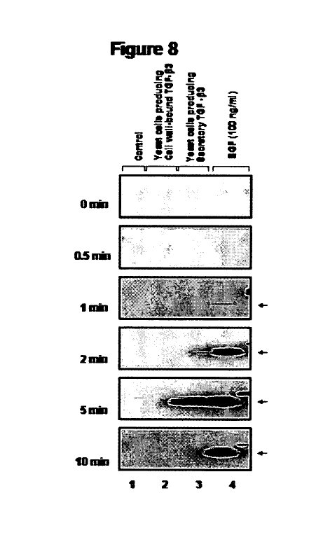

As many mitogens trigger phosphorylation of Erk protein, leading to transduction of an activation signal to downstream molecules, measurement of phospho-Erk levels can be used to monitor activation of signal transduction cascades. Phosphorylation of Erk was observed 1 to 10 min after RINm5F cells were treated with the positive control, purified epidermal growth factor (EGF) (FIG. 8, lane 4). Phosphorylation of Erk was also observed following co-culture of RINm5F cells with yeasts expressing secretory zymo-sTGF-p3 (FIG. 8, lane 3). In contrast, Erk phosphorylation was not observed following co-culture of cells with yeasts expressing cell wall-bound zymo-bTGF-β3 (FIG. 8, lane 2) or the negative control plasmid (FIG. 8, lane 1). This indicates that short-term treatment of mammalian cells with yeasts expressing TGF-β3 can trigger signal transduction cascades, and that measurement of protein phosphorylation is another method for assessing zymogand activity in the inventive system.

### REFERENCES

1. Bartenschlager, R. 2002. Hepatitis replicons: potential role for drug development. Nat Rev Drug Discov. 1: 911-916.
2. Ram A F, Van den Ende H, Klis F M. 1998. Green fluorescent protein-cell wall fusion proteins are covalently incorporated into the cell wall of Saccharomyces cerevisiae. FEMS Microbiol Lett. 162(2):249-55.
3. Smith, G. P. 1985. Filamentous fusion phage: novel expression vectors that display cloned antigens on the virion surface. Science 228: 1315-1317.
4. Van der Vaart J M, Biesebeke R, Chapman J W, Toschka H Y, Klis F M, Verrips C T. 1997. Comparison of cell wall proteins of Saccharomyces cerevisiae as anchors for cell surface expression of heterologous proteins. Appl Environ Microbiol. 63(2): 615-20.
5. Vrolijk, J M, Kaul, A, Hansen, B E, Lohman, V, Haagmans, B L, Schalm, S W, Bartenschlager, R. 2003. J Virol Methods. 110: 201-209.
6. Schreuder M P, Deen C, Boersma W J, Pouwels P H, Klis F M. 1996 Yeast expressing hepatitis B virus surface antigen determinants on its surface: implications for a possible oral vaccine. Vaccine 14,383-388.
7. Boder, E. T. and Wittrup, K. D. 1997 Yeast surface display for screening combinatorial polypeptide libraries. Nat. Biotechnol. 15, 553-557.
8. Murai T, Ueda M, Yamamura M, Atomi H, Shibasaki Y, Kamasawa N, Osumi M, Amachi T, Tanaka A. 1997 Construction of a starch-utilizing yeast by cell surface engineering. Appl Environ Microbiol. 63(4):1362-1366.
9. Washida M, Takahashi S, Ueda M, Tanaka A. 2001 Spacer-mediated display of active lipase on the yeast cell surface. Appl Microbiol Biotechnol. 56(5-6):681-686.
10. Ye K, Shibasaki S, Ueda M, Murai T, Kamasawa N, Osumi M, Shimizu K, Tanaka A. 2000 Construction of an engineered yeast with glucose-inducible emission of green fluorescence from the cell surface. Appl Microbiol Biotechnol. 54(1):90-96.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A polypeptide assay system comprising: a non-mammalian cell in a non-mammalian cell culture medium expressing a heterologous polypeptide that is either displayed on its cell surface such that the polypeptide is the predominant polypeptide displayed on the cell surface or the polypeptide is secreted; and a target mammalian cell comprising a reporter construct in a mammalian cell culture medium.

2. The assay system according to claim 1, wherein the non-mammalian cell culture medium is not suitable for culturing mammalian cell, and the mammalian cell culture medium is suitable for culturing mammalian and non-mammalian cell.

3. The assay system according to claim 1, wherein the non-mammalian cell and the mammalian cell are mixed together.

4. The assay system according to claim 1, wherein the non-mammalian cell is a fungal cell or prokaryotic cell.

5. The assay system according to claim 1, wherein the fungal cell is yeast cell.

6. The assay system according to claim 4, wherein the yeast cell belongs to the genus Saccharomyces.

7. The assay system according to claim 1, wherein the non-mammalian cell is a conditional mutant.

8. The assay system according to claim 7, wherein the non-mammalian cell is temperature sensitive.

9. A method of assaying for the function of a polypeptide comprising:
(i) culturing a non-mammalian cell expressing a heterologous polypeptide in a non-mammalian cell culture medium so that the polypeptide is displayed on the cell surface such that the polypeptide is the predominant polypeptide displayed on the cell surface;
(ii) culturing a target mammalian cell comprising a reporter construct in a mammalian cell culture medium;
(iii) mixing the non-mammalian cell culture in (a) with the mammalian cell culture in (b), wherein a change in expression of the reporter construct in the mammalian cell indicates that the heterologous polypeptide is a modulator of the reporter.

10. The method according to claim 9, wherein the non-mammalian cell culture medium is not suitable for culturing mammalian cell, and the mammalian cell culture medium is suitable for culturing mammalian and non-mammalian cell.

11. The method according to claim 9, wherein the non-mammalian cell is a fungal cell or prokaryotic cell.

12. The method according to claim 11, wherein the fungal cell is yeast cell.

13. The method according to claim 12, wherein the yeast cell belongs to the genus Saccharomyces.

14. The method according to claim 9, wherein the non-mammalian cell is a conditional mutant.

15. The method according to claim 14, wherein the non-mammalian cell is temperature sensitive.

16. The method according to claim 15, wherein the temperature of the mixed culture medium is modified so that the mammalian cell grows but the non-mammalian cell does not grow in the medium.

17. A method of assaying for the function of a polypeptide comprising:
(i) culturing a non-mammalian cell expressing a heterologous polypeptide in a culture medium so that the polypeptide is secreted;
(ii) culturing a target mammalian cell comprising a reporter construct;
(iii) mixing the non-mammalian cell culture medium comprising the secreted polypeptide in (a) with the mammalian cell culture in (b), wherein a change in expression of the reporter construct in the mammalian cell indicates that the heterologous polypeptide is a modulator of the reporter.

18. The method according to claim 17, wherein the non-mammalian cell is a fungal cell or prokaryotic cell.

19. The method according to claim 18, wherein the fungal cell is yeast cell.

20. The method according to claim 19, wherein the yeast cell belongs to the genus Saccharomyces.
